(19) United States
(12) Patent Application Publication
(10) Pub. No.: US 2005/0112095 A1
(43) Pub. Date: May 26, 2005

(54) Internal ribosome entry sites for recombinant protein expression

(76) Inventors: Tsu-An Hsu, Taipei (TW); Tzong-Yuan Wu, Panchiao City (TW); Jin-Ching Lee, Taichung (TW)

Correspondence Address: Finnegan, Henderson, Farabow,;Garrett & Dunner, L.L.P., 1300 I Street, N.W., Washington, DC, 20005-3315, US

(21) Appl. No.: 10/614,283

(22) Filed: Jul. 8, 2003

Related U.S. Application Data
(60) Provisional application No. 60/394,269, filed on Jul. 9, 2002.

(51) Int. Cl. A61K 48/00; C12N 15/86; C12N 15/861; C12N 15/867

(52) U.S. Cl. 424/93.2; 435/456

(57) ABSTRACT

The invention describes compositions and methods for recombinant protein expression in a wide range of cell types, including mammalian, insect, and bacterial cells. The compositions comprise a viral IRES sequence selected from enterovirus 71 (EV71), hepatitis C virus (HCV), or encephalomyocarditis virus (EMCV), or a variant or fragment thereof, or alternatively, a homolog of a viral IRES selected from EV71, HCV, or EMCV, or a variant or fragment thereof. Methods of using the compositions are also described.

### FIELD OF THE INVENTION

The present invention relates to the 5′ untranslated regions (5′UTRs) of viral genes which function as internal ribosome entry sites (IRESs). In particular, the present invention relates to the IRES of encephalomyocarditis virus (EMCV), Hepatitis C virus (HCV), and Enterovirus 71 (EV71). The present invention further relates to methods of using the various IRESs in recombinant protein expression systems, to compositions comprising the various IRESs, and to methods of screening for anti-viral compounds using the IRESs of the present invention.

### BACKGROUND OF THE INVENTION

Eukaryotic mRNAs have a distinctive structural feature at their 5′ end, called a 5′ cap, which is a residue of 7-methylguanosine linked to the 5′ terminal residue of the mRNA through an unusual 5′,5′-triphosphate linkage. Cap-dependent translation is initiated by the binding of the cap-binding protein complex eIF-4F to the 5′ cap, which in turn facilitates the binding of the 43S ternary ribosomal subunit near or at the 5′ cap region. The ribosome complex is purported to scan the mRNA from the 5′ cap until it encounters the first AUG initiation codon, where translation of the mRNA is initiated. (see Kozak, M, (1989) Cell 44:283-292; Kozak, M (1989) J. Cell. Biol. 108:229-241).

A cap-independent translation mechanism was proposed to explain the efficient translation of some mRNAs despite the presence of a highly ordered RNA structure in 5′ untranslated region (5′UTR) of mRNAs which was predicted to interfere with ribosome scanning of the mRNA. The picornavirus mRNA was the first mRNA identified that displayed a cap-independent translation mechanism (Jackson, R. J., (1988) Nature 334:292-293). The picornavirus mRNA is characterized by a unique structure, including the absence of a 5′ cap, the presence of an extraordinarily long and structured 5′ UTR, and the presence of multiple upstream AUG initiation codons. This long and structured 5′UTR was found to serve as an internal ribosome entry site (IRES) or a ribosome landing pad, where the 43S ternary ribosomal subunit would bind and initiate translation independently of the 5′ cap structure.

The 5′UTR containing an IRES is generally characterized by three complex features: a long 5′UTR, a stable secondary structure, and potential upstream AUG initiation codons. The stable secondary structure is considered to be the major determinant of IRES function. A low proportion of vertebrate mRNAs have long, highly structured 5′UTRs that contain multiple AUG initiation codons. Among these, the Drosophila Antp gene has been found to harbor a 1,735 nt-long 5′UTR and 15 upstream AUG codons, and the Ubx gene has a 968 nt-long 5′UTR and two upstream AUG codons. To date, a limited, but a growing subset of IRESs have been identified in cellular mRNAs in various species including human (Macajak, D. G. and P. Sarnow, (1991) Nature 353:653-656; Sarnow, P, (1989) PNAS 86:5795-5799; Vagner, S. et al., (1995) Mol. Cell. Biol. 15:35-44), and yeast (Zhou, W. et al., (2001) PNAS 98:1531-1536; Paz, I. et al., (1999) J. Biol. Chem. 274:21741-21745). IRESs have also been identified in viral mRNAs, such as in poliovirus (Pelletier, J. and N. Sonenberg. (1988) Nature 334:320-325), encephalomyocarditis virus (EMCV) (Jang, S. K., and E. Wimmer, (1990) Genes Dev. 4:1560-1572), and human rhinovirus (HRV) (Borman, A. et al., (1993) J. Gen. Virol. 74:1775-1788). The Antp and Ubx homeotic genes of Drosophila are also translated via an IRES in their long 5′UTRs (Ye X. et al., (1997) Mol. Cell. Biol. 17:1714-1721; Ho, S.-K. et al., (1992) Genes Dev. 6:1643-1653).

### SUMMARY OF THE INVENTION

The present invention provides an internal ribosomal entry site (IRES) from the 5′ UTR of the enterovirus 71 (EV71) gene. The enterovirus is a genus of the family Picornaviridae and the enterovirus 71 is a member of the enterovirus genus (see Fields, B. N., et al., eds., (3^{rd} ed. 1996) Fundamental Virology, Lippincott-Raven, Philadelphia, Pa., p. 477-522). The activity of the EV71 IRES is compared to those of the encephalomyocarditis virus (EMCV) and Hepatitis C virus (HCV) IRESs. All of these viral IRESs direct the cap-independent translation of mRNA in various cell types, including mammalian, insect, and bacterial cells. Thus, the viral IRESs are useful in nucleic acid vectors to direct the expression of two or more unrelated proteins from a single transcriptional unit.

Conventionally, a recombinant protein is expressed in a cell by placing its gene under the control of a promoter, which provides the RNA polymerase binding site necessary for mRNA synthesis. When two or more recombinant proteins are to be expressed in a cell, each of their genes is placed under the control of separate promoters in a single nucleic acid vector. Alternatively, each of the proteins may be expressed from separate nucleic acid vectors. In either method, a separate mRNA transcript is generated for each protein. Translation of different mRNA transcripts often leads to the uncoupled expression of the various proteins. If multiple proteins are placed under the control of a single promoter, it has been observed that the first gene most proximal to the 5′ cap is most efficiently translated, presumably by the cap-dependent process, while the downstream genes may be translated at low levels or not at all. However, when an IRES is inserted into a nucleic acid vector between genes downstream of the 5′ most proximal gene, two or more proteins may be efficiently translated from a single mRNA transcript.

The nucleic acid vector directing the expression of more than one protein from a single vector is known in the art as a multicistronic vector. In a multicistronic vector, a nucleotide sequence comprising at least two cistrons, or genes, is placed under the control of a promoter for mRNA synthesis, and an IRES is inserted between two cistrons. A single mRNA transcript is generated containing sequences of the first cistron, IRESs, and other downstream cistrons, rather than separate mRNA transcripts as in the conventional approach. During translation, the first cistron is translated by the ribosomal scanning mechanism because it is most proximal to the 5′ cap while the second cistron and other downstream cistrons are translated by internal ribosome binding to the IRES. As a result, a constant ratio of mRNAs expressing multiple cistrons is maintained. The major advantage of this technique is the co-expression of two or more proteins from a single mRNA, avoiding the use of separate expression constructs and multiple promoters which often leads to uncoupled expression of the proteins.

The viral IRESs disclosed in the present invention can direct such cap-independent translation in a wide range of cell types, including insect, mammalian, and bacterial cells. This is quite advantageous because the baculovirus expression system is widely applicable for the high level production of recombinant proteins. Many biologically active proteins have been produced at high levels using the baculovirus system (for review see Miller, L. K., (1988) Annu. Rev. Microbiol. 42:177-199; Luckow V. A. and M. D. Summers, (1988) Bio/Technology 6:47-55; Luckow V. A., (1990) In: Recombinant DNA Technology and Applications. McGraw-Hill, New York, pp. 97-152; O'Reilly, D. R., et al., (1992) Baculovirus Nucleic Acid Vectors: A Laboratory Manual. W.H. Freedman, New York). In the baculovirus system, the baculovirus polyhedrin gene is usually replaced with the gene encoding for the protein of interest. The polyhedrin gene is highly expressed in infected insect cells but is not essential for viral propagation, and is therefore the ideal location to place the gene of interest. This segment of the baculovirus gene is placed in a separate transfer vector and under the control of a strong polyhedrin promoter or other baculovirus promoter. This transfer vector is co-transfected into baculovirus host cells with a baculovirus genomic DNA. Recombinant baculoviruses carrying the gene of interest is produced when homologous recombination between the transfer vector and baculovirus genomic DNA occurs. These recombinant baculoviruses are used to infect host cells, which will produce large amounts of the desired protein.

However, despite the attractiveness of the baculovirus expression system, other IRESs have not been shown to be active in baculovirus host cells. Thus, while the encephalomyocarditis virus (EMCV) IRES element is known to be highly efficient in mammalian systems, the literature reports that it does not promote efficient internal translation in various baculovirus host insect cells, presumably because the insect cells do not have the cellular factors required to initiate internal translation that are present in mammalian cells (Finkelstein Y., et al., (1999) J. Biotech. 75:33-44).

Contrary to the above reports, the inventors have surprisingly found that the EMCV IRES element functions in baculovirus host insect cells. The inventors have also found other IRESs that function in baculovirus host insect cells as well as in other cell types, including mammalian and bacterial cells. Thus, the present invention provides a kit for recombinant protein expression in bacteria, insect, and/or mammalian cells comprising at least one nucleic acid vector comprising at least one IRES sequence functional in a bacterial cell, at least one nucleic acid vector comprising at least one IRES sequence functional in a insect cell, and at least one nucleic acid vector comprising at least one IRES sequence function in a mammalian cell.

The present invention also provides homologs, fragments, and variants of the IRESs of EV71, HCV, and EMCV, as well as variants and fragments of homologs of the EV71, HCV, and EMCV IRESs. The present invention further provides multicistronic nucleic acid vectors comprising a viral IRES disclosed in the present invention or a homolog, fragment, or variant thereof having IRES activity, for the production of multiple recombinant proteins from a single mRNA transcript. These multicistronic nucleic acid vectors may be contained in a biological vector capable of expressing multiple genes in a host cell. These nucleic acid vectors and biological vectors may be used for the genetic treatment in patients and/or the recombinant proteins produced thereby may be useful as therapeutic agents.

The present invention also provides a baculovirus transfer vector and a recombinant baculovirus for the expression of at least two genes in a baculovirus host cell, comprising a viral IRES disclosed in the present invention or a homolog, variant, or a fragment thereof having IRES activity. The ability to express two or more genes from a single baculovirus transfer vector and a recombinant baculovirus greatly simplifies the process of isolating plaques expressing the gene(s) of interest. Moreover, the expression of a gene of interest and a reporter gene would also allow the simultaneous evaluation of recombinant protein level produced and the detection/isolation of cells producing the recombinant protein.

The present invention further provides a method of screening for anti-viral compounds which interfere with cap-independent translation from the viral IRES. The method comprises transfecting a nucleic acid vector which directs the cap-independent translation of a recombinant protein into a cell, contacting the transfected cell with a test compound, and detecting a decrease in recombinant protein production compared to a cell without the test compound.

### BRIEF DESCRIPTION OF THE DRAWINGS

FIG. 1 FIG. 1 shows the nucleotide sequence of the EV71 5′UTR from an EV71 gene of strain TW/2086/98.

FIG. 2 FIGS. 2A and 2B show schematic diagrams of a recombinant baculovirus transfer vector, pBac-EGFP, used to generated a recombinant baculovirus.

FIG. 2C shows EGFP expression in Sf9 cells infected with the recombinant baculovirus as observed under fluorescent microscopy.

FIG. 3 FIGS. 3A and 3B show schematic diagrams of a recombinant baculovirus transfer vector, pBac-IR-EGFP, in which the EMCV IRES immediately precedes the EGFP coding sequence. FIG. 3C shows EGFP expression in Sf9 cells infected with the recombinant baculovirus as observed under fluorescent microscopy.

FIG. 4 FIG. 4 shows schematic diagrams of a recombinant baculovirus transfer vector, pBac-DR-IR-EGFP, in which the DsRed and EGFP coding sequences are placed under the control of the polyhedrin promoter for mRNA synthesis, and the EMCV IRES is placed between the DsRed and EGFP coding sequences to drive the cap-independent translation of EGFP.

FIG. 5 FIG. 5 shows Sf9 cells infected with a recombinant baculovirus carrying pBac-DR-IR-EGFP as observed under fluorescent microscopy. The left panel shows cells expressing DsRed, and the right panel shows cells expressing EGFP.

FIG. 6 FIG. 6 shows a schematic diagram of the bicistronic nucleic acid vector used for expression of the β-galactosidase (β-gal) and secreted alkaline phosphatase (SEAP) genes in mammalian, insect, and bacterial cells. The EV71, HCV, or EMCV IRES sequences were inserted between the β-gal and SEAP genes to drive the cap-independent translation of SEAP. The respective bicistronic nucleic acid vectors were designated pGS-EV71, pGS-HCV, and pGS-EMCV.

FIG. 7 FIG. 7 shows the activity of EMCV, HCV, and EV71 IRESs in Sf9 insect cells. The Sf9 insect cells were infected with recombinant baculoviruses generated from transfer vectors pGS-EMCV, pGS-HCV, and pGS-EV71.

FIG. 8 FIG. 8 shows the activity of EMCV, HCV, and EV71 IRESs in COS-7 and Huh7 cells.

FIG. 9 FIG. 9 shows IRES activity in BL21 cells. Cells analyzed were untransformed BL21 cells (lane 1), BL21 cells transformed with pTriEX-4 containing no reporter gene (lane 2), cells transformed with pGS-EMCV and without IPTG induction (lane 3), cells transformed with pGS-EMCV and induced with 0.4 mM IPTG (lane 4), cells transformed with pGS-HCV and induced with 0.4 mM IPTG (lane 5), and cells transformed with pGS-EV71 and induced with 0.4 mM IPTG (lane 6).

FIG. 10 FIG. 10 is an illustration of the process involved in screening for anti-viral compounds that interfere with cap-independent translation from a viral IRES using a multicistronic nucleic acid vector.

FIG. 11 FIG. 11 shows the anti-viral activity of interferon-alpha (IFN-α) on HCV IRES.

FIG. 12 FIG. 12 shows the anti-viral activity of interferon-alpha (IFN-α) on EV71 IRES.

### DETAILED DESCRIPTION OF THE INVENTION

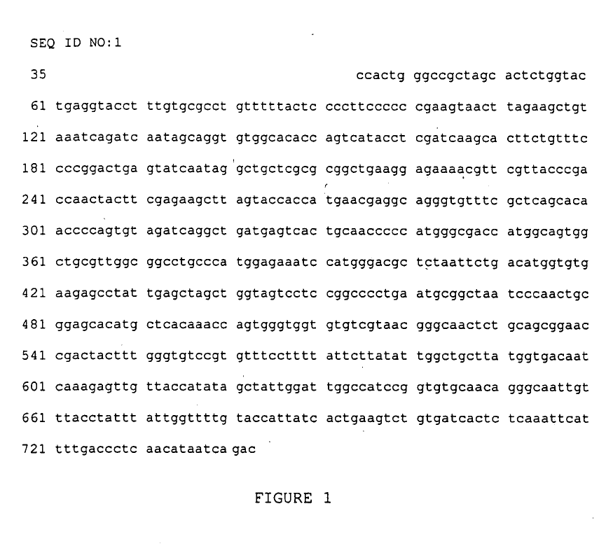

The present invention provides an isolated nucleotide sequence or cDNA of the internal ribosome entry site (IRES) in the 5′UTR region of the enterovirus 71 (EV71). The 5′ untranslated region (UTR) of the EV71 gene is about 700 nucleotides in length. An example of a EV71 5′UTR from an EV71 gene (strain TW/2086/98) is set forth in SEQ ID NO:1 and in FIG. 1. An isolated nucleotide sequence or cDNA of the invention may be isolated by any technique known in the art, for example, by cloning using suitable probes, by the polymerase chain reaction (PCR), or alternatively, by chemical synthesis. As shown hereinbelow, the 5′UTR of the EV71 gene exhibits IRES activity. Other viral IRESs are known in the art. For example, the encephalomyocarditis virus (EMCV) IRES, is disclosed in Jang, S. K., and E. Wimmer, (1990) Genes Dev. 4:1560-1572. The hepatitis C virus (HCV) IRES is about 332 or 341 nucleotides long, depending on specific virus strains (Tsukiyama-Kohara K., et al., (1992) J. Virol. 66:1476-1483; Buratti E., et al., (1997) FEBS Lett. 411:275-280).

As used herein, “IRES activity” refers to cap-independent translation initiated by internal ribosome binding, as opposed to cap-dependent translation. “Cap-dependent translation” refers to the mechanism of translation in which the ribosomal unit essential for initiating translation binds to mRNA at or near the 5′ cap region on the mRNA. Cap-dependent translation is purported to proceed by a “ribosome scanning” mechanism whereby the ribosome complex scans the mRNA from the 5′ cap until it encounters an AUG initiation codon. “Cap-independent translation” refers to the mechanism of translation in which the ribosomal unit essential for initiating translation binds to a site on the mRNA without requiring the 5′ cap region. As used herein, the “IRES” is a nucleotide sequence that provides a site for ribosomal binding for cap-independent translation.

The present invention also relates to homologs, variants, or fragments of the EV71, HCV, and EMCV IRESs.

As used herein, “homolog” refers to structures or processes in different organisms that show a fundamental similarity. A homolog of the EV71, HCV, or EMCV IRES may have a primary or secondary structure similar to the EV71, HCV, or EMCV IRES, respectively, and/or have IRES activity. Secondary structure may be predicted using computer programs known in the art, such as Zuker's RNA folding program (Zuker, M., (1989) Methods Enzymol. 108:262-288). The present invention also includes variants and fragments of homologs of the EV71, HCV, and EMCV IRESs.

As used herein, “variant” of EV71, HCV, or EMCV IRES refers to a naturally-occurring or synthetically produced nucleotide sequence substantially identical to that of the EV71, HCV, or EMCV IRES, respectively, but which has a nucleotide sequence different from that of the EV71, HCV, or EMCV IRES because of one or more deletions, substitutions, or insertions. A variant of EV71, HCV or EMCV IRES retains IRES activity or has enhanced IRES activity compared with the EV71, HCV, or EMCV IRES, respectively.

As used herein, “fragment” of EV71, HCV, or EMCV IRES refers to a portion of the IRES nucleotide sequence that comprises less than the complete IRES nucleotide sequence and that retains essentially the same or exhibits enhanced IRES activity as the complete IRES nucleotide sequence.

Sequence “similarity” and/or “identity” are used herein to describe the degree of relatedness between two polynucleotides or polypeptide sequences. In general, “identity” means the exact match-up of two or more nucleotide sequences or two or more amino acid sequences, where the nucleotide or amino acids being compared are the same. Also, in general, “similarity” means the exact match-up of two or more nucleotide sequences or two or more amino acid sequences, where the nucleotide or amino acids being compared are either the same or possess similar chemical and/or physical properties. The percent identity or similarity can be determined, for example, by comparing sequence information using the GAP computer program, version 6.0 described by Devereux et al. (Nucl. Acids Res. 12:387, 1984) and available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math 2:482, 1981). Other programs for calculating identity and similarity between two sequences are known in the art.

For purposes of the invention, a homolog, variant, or fragment of the EV71, HCV, or EMCV IRES may exhibit at least about 20% nucleotide identity with the EV71, HCV, or EMCV IRES, respectively, at least about 30% nucleotide identity, or at least about 40% nucleotide identity, although the invention certainly encompasses sequences that exhibit at least about 50%, 60%, 70%, 80% and 90% nucleotide identity with EV71, HCV, or EMCV IRES. Furthermore, a homolog, variant, or fragment of the EV71, HCV, or EMCV IRES may exhibit a similar range of nucleotide sequence similarity with the EV71, HCV, or EMCV IRES, respectively, from at least about 50%, 60%, 70%, 80%, and 90% nucleotide sequence similarity. Similarly, variants or fragments of the EV71, HCV, or EMCV IRES homolog may exhibit a nucleotide identity with the EV71, HCV, or EMCV IRES homolog, respectively, of at least about 20% up to at least about 90% in increments of 10 as above, or a nucleotide similarity with the EV71, HCV, or EMCV IRES homolog of at least about 50% to at least about 90%, in increments of 10 as above. Naturally-occurring homologs, variants, and fragments are encompassed by the invention.

Homologs, variants, or fragments of EV71, HCV or EMCV IRES may be obtained by mutation of nucleotide sequences of the EV71, HCV, or EMCV IRES, respectively, following techniques that are routine in the art. Mutations may be introduced at particular locations by synthesizing oligonucleotides containing a mutant sequence, flanked by restriction sites enabling ligation to fragments of the native sequence. Following ligation, the resulting reconstructed sequence contains the desired insertion, substitution, or deletion. See Sambrook et al., Molecular Cloning: A Laboratory Manual, Vols 1-3 (2d ed. 1989), Cold Spring Harbor Laboratory Press.

Alternatively, oligonucleotide-directed site-specific mutagenesis procedures may be employed to provide an altered nucleotide sequence wherein predetermined sequences may be altered by substitution, deletion or insertion. Exemplary methods of making the alterations set forth above are known in the art (Walder R. Y. et al., (1986) Gene 42:133-139; Bauer C. E., et al., (1985) Gene 37:73-81; Craik C. S., (Jan. 1985) BioTechniques, 12-19; Smith et al., (1981) Genetic Engineering: Principles and Methods, Plenum Press; Kunkel T. A., (1985) Proc. Natl. Acad. Sci. USA 82:488-492; Kunkel T. A., et al., (1987) Methods in Enzymol. 154:367-382; U.S. Pat. Nos. 4,518,584 and 4,737,462, all of which are incorporated by reference). Other methods known in the art may also be used.

IRES activity may be determined by its ability to translate mRNA independently of the 5′ cap region of the mRNA. Several reports support the hypothesis that IRES activity is cell type-dependent (Oumard A., et al., (2000) Mol. Cell. Biol. 20:2755-2759; Stoneley M., et al., (1998) Oncogene 16:423-428; Pozner A., et al., (2000) Mol. Cell. Biol. 20:2297-2307). These reports suggested that IRES activity is dependent on interaction with specific protein factors present in different cells.

The EV71, HCV, and EMCV IRES or a homolog, variant, or fragment thereof of the present invention is capable of directing cap-independent translation in various cell types, including mammalian, bacterial, and insect cells. The EV71, HCV, and EMCV IRES or a homolog, variant, or fragment thereof of the present invention may also have IRES activity in other eukaryotic cells, such as yeast and plants.

The present invention further encompasses DNA constructs comprising the EV71, HCV, or EMCV IRES, or a homolog, variant, or fragment thereof, such as plasmids and recombinant expression vectors. In recombinant expression vectors, the EV71, HCV, or EMCV IRES or a homolog, variant, or fragment thereof directs the expression of at least one recombinant protein. The construction and expression of conventional recombinant nucleic acid vectors is well known in the art and includes those techniques contained in Sambrook et al., Molecular Cloning: A Laboratory Manual, Vols 1-3 (2d ed. 1989), Cold Spring Harbor Laboratory Press. Such nucleic acid vectors may be contained in a biological vector such as viruses and bacteria, preferably in a non-pathogenic or attenuated microorganism, including attenuated viruses, bacteria, parasites, and virus-like particles.

In the context of the present invention, the nucleotide sequence of the EV71, HCV, or EMCV IRES or a homolog, variant, or fragment thereof is positioned upstream of a gene, or cistron, of interest in the nucleic acid vector in order to direct the cap-independent translation of an expression product. A variant or fragment of an EV71, HCV or EMCV IRES homolog may also be used. The nucleic acid vector may be of the monocistronic type (for the expression of a single gene of interest under the control of a promoter for mRNA synthesis) or of the multicistronic type (for the expression of at least two genes of interest placed under the control of the same promoter for mRNA synthesis). Such a nucleic acid vector may contain several “IRES-cistron” elements in tandem, wherein at least one of the IRES sites comprises the nucleotide sequence of the EV71, HCV, or EMCV IRES or a homolog, variant, or fragment thereof, or alternatively, a variant or fragment of an EV71, HCV, or EMCV IRES homolog.

The nucleic acid vectors of the present invention comprise a promoter operably linked to a nucleotide sequence comprising at least one cistron operably linked to a nucleotide sequence of an EV71, HCV, or EMCV IRES or a homolog, variant, or fragment thereof, or a variant or fragment of an EV71, HCV, or EMCV IRES homolog. A promoter is required for mRNA synthesis from a DNA sequence and an mRNA with a 5′ cap is usually synthesized in eukaryotes. As used herein, “cistron” refers to a polynucleotide sequence, or gene, of a protein, polypeptide, or peptide of interest. “Operably linked” refers to a situation where the components described are in a relationship permitting them to function in their intended manner. Thus, for example, a promoter “operably linked” to a cistron is ligated in such a manner that expression of the cistron is achieved under conditions compatible with the promoter. Similarly, a nucleotide sequence of an IRES operably linked to a cistron is ligated in such a manner that translation of the cistron is achieved under conditions compatible with the IRES. The nucleic acid vector may further comprise one or more additional “IRES-cistron” elements in tandem.

Cistrons may include genes coding for receptors, ion channels, subunits of proteins, enzymes, antibodies, protein ligands, proteins conferring antibiotic resistance to cells, growth factors, hormones, or any other proteins, polypeptides, or peptides of interest. In one embodiment of the present invention, at least one cistron in the nucleic acid vector of the present invention comprises a therapeutic gene coding for a therapeutic agent capable of inhibiting or delaying the establishment and/or development of a genetic or acquired disorder, such as cystic fibrosis, hemophilia A or B, Duchenne or Becker type myopathy, cancer, AIDS and other bacteria or infectious diseases due to a pathogenic organism. Examples of such therapeutic agents include, but are not limited to: a cytokine; interleukin; interferon; a factor or cofactor involved in coagulation, such as factor VIII, factor IX, von Willebrand factor, antithrombin III, protein C, thrombin, and hirudin; enzyme inhibitors such as viral protease inhibitors; an ion channel activator or inhibitor; a protein capable of inhibiting the initiation or progression of cancers, such as expression products of tumor suppressing genes (p53, Rb genes, etc.), a toxin, an antibody, or an immunotoxin; or a protein capable of inhibiting a viral infection or its development, for example, an antigenic epitope of the virus in question, an antibody or an altered variant of a protein capable of competing with the native viral protein.

In another embodiment of the present invention, at least one cistron in the nucleic acid vector of the present invention comprises a reporter gene, for example, a gene coding for β-galactosidase, firefly luciferase, green fluorescent protein, the red fluorescent protein from Discosoma sp. (DsRed), or secreted alkaline phosphatase (SEAP). Other reporter genes known in the art may be used. Reporter genes facilitate the detection of cells expressing a functional protein from a nucleic acid vector. Detection of reporter proteins may be made by providing a substrate required for the enzymatic reaction producing a readily detectable product by eye, luminescence, fluorescence, or microscopy. Other reporter gene products, such as the green fluorescent protein, may be observed directly under the microscope under appropriate fluorescent or luminating conditions.

Promoters that may be sued in the invention include viral promoters and cellular promoters and are well known in the art. Viral promoters may include the cytomegalovirus (CMV) promoter, the baculovirus polyhedrin promoter, the major late promoter from adenovirus 2 and the SV40 promoter. Examples of cellular promoters include the Drosophila actin 5C distal promoter and the mouse metallothionein 1 promoter. Other promoters useful for the nucleic acid vectors of the present invention may be readily determined by those skilled in the art.

Also contained in nucleic acid vectors is a polyadenylation signal located downstream of the last cistron of interest. Polyadenylation signals include the early or late polyadenylation signals from SV40, adenovirus 5 E1B, and the human growth hormone gene. The nucleic acid vectors may also include an enhancer sequence, such as the SV40 and CMV enhancer.

In order to identify cells that have acquired the nucleic acid vector, a selectable marker is generally introduced into the cells along with the gene of interest. Selectable markers include genes that confer drug resistance to the cells, such as ampicillin, neomycin, hygromycin and methotrexate. Selectable markers are reviewed by Thilly (Mammalian Cell Technology, Butterworth Publishers, Stoneham, Mass.) and the choice of selectable markers is well within the level of ordinary skill in the art.

Selectable markers may be introduced into the cell on a separate plasmid at the same time as the nucleic acid vector or they may be on the same nucleic acid vector. If on the same nucleic acid vector, the selectable marker and gene(s) of interest may be under the control of different promoters or IRESs or the same promoter or IRES.

If it is desired that the gene product of interest be secreted from the cell, a secretory signal sequence may be placed immediately upstream of and in-frame of the gene of interest in the nucleic acid vector. Many secretory signal sequences are known in the art, such as the signal sequences of human serum albumin, human growth factor, the alpha factor signal sequence, and the immunoglobulin chains, to name a few. Alternatively, secretory signal sequences may be synthesized according to the rules established, for example, by von Heinje (Eur. J. Biochem. 13: 17-21,1983; J. Mol. Biol. 184:99-105,1985; Nuc. Acids Res. 14:4683-4690,1986).

The present invention also encompasses methods for expressing at least one cistron of interest by a cap-independent process comprising introducing into a host cell a nucleic acid vector comprising a promoter operably linked to a nucleotide sequence comprising at least one cistron operably linked to a nucleotide sequence of an EV71, HCV, or EMCV IRES or a homolog, variant, or fragment thereof, or a variant or fragment of an EV71, HCV, or EMCV IRES homolog. The nucleic acid vector may further comprise one or more additional “IRES-cistron” elements in tandem for expression of at least two cistrons by a cap-independent process.

The nucleic acid vectors may be introduced into cultured host cells by, for example, calcium phosphate-mediated transfection (Wigler et al., (1978) Cell 14:725; Corsaro and Pearson (1981) Somatic Cell Genetics 7:603; Graham and Van der Eb. (1973) Virology 52:456). Other techniques for introducing nucleic acid vectors into host cells, such as electroporation (Neumann et al., (1982) EMBO J. 1:841-845), may also be used.

Transfected cells are allowed to grow for a period of time to allow the expression of the gene(s) of interest. Drug selection may be applied to select for growth of cells expressing the selectable marker. Host cells containing the nucleic acid vectors of the present invention are grown in an appropriate growth medium. As used herein, the term “appropriate growth medium” means a medium containing nutrients required for the growth of cells. Nutrients required for cell growth may include a carbon source, a nitrogen source, essential amino acids, vitamins, minerals and growth factors. The growth medium may also include a drug to select for cells expressing a selectable marker from the introduced nucleic acid vector.

A stable cell line may be established when the cells have been selected for stable integration of the gene of interest into the host genome. Usually, stable cell lines are established after having undergone drug selection for about three days to about three weeks.

As discussed above, the present invention provides IRES sequences that are active in a wide range of cell types, including bacteria, insect, and/or mammalian cells. Thus, the present invention relates to a kit for recombinant protein expression in bacteria, insect, and/or mammalian cells comprising at least one nucleic acid vector comprising at least one IRES sequence functional in a bacterial cell, at least one nucleic acid vector comprising at least one IRES sequence functional in a insect cell, and at least one nucleic acid vector comprising at least one IRES sequence functional in a mammalian cell. In an embodiment of the present invention, the kit comprises at least one nucleic acid vector comprising at least one EV71 IRES sequence, at least one nucleic acid vector comprising at least one HCV IRES sequence, and at least one nucleic acid vector comprising at least one EMCV IRES sequence. In another embodiment, the kit comprises a single nucleic acid vector comprising at least one IRES sequence functional in bacteria, insect, and mammalian cells. In yet another embodiment of the present invention, the kit comprises two nucleic acid vectors wherein said two nucleic acid vectors each comprise at least one IRES sequence functional in bacteria, insect, and/or mammalian cells.

As described above, the nucleic acid vector of the present invention may be contained in a biological vector such as viruses and bacteria, preferably in a non-pathogenic or attenuated microorganism, including attenuated viruses, bacteria, parasites, and virus-like particles. Examples of such biological vectors include poxvirus (e.g. vaccinia virus), adenovirus, baculovirus, herpesvirus, adeno-associated virus, and retrovirus. Such vectors are amply described in the literature. In an embodiment of the present invention, the nucleic acid vector of the present invention may be contained in a recombinant baculovirus capable of infecting a baculovirus host cell and expressing a gene of interest. The baculovirus expression system is described in the art, for example, in U.S. Pat. Nos. 4,745,051, 4,879,236, and 5,147,788, Miller, L. K., (1988) Annu. Rev. Microbiol. 42:177-199; Luckow, V. A., (1990) In: Recombinant DNA Technology and Applications. McGraw-Hill, New York, pp. 97-152; and O'Reilly, D. R., et al., (1992) Baculovirus Nucleic acid vectors: A Laboratory Manual. W.H. Freedman, New York, all of which are incorporated herein by reference.

In general, generation of recombinant baculoviruses capable of infecting a host cell and expressing a gene of interest involves the co-transfection of a recombinant transfer vector and a baculovirus genomic DNA into a baculovirus host cell. A recombinant baculovirus transfer vector is generally derived from a DNA fragment of the baculovirus genomic DNA comprising the polyhedrin promoter and polyhedrin gene. In a recombinant baculovirus transfer vector, a gene of interest is placed under the control of the polyhedrin promoter or other baculovirus promoter, replacing some or all of the sequences of the polyhedrin gene. A recombinant baculovirus transfer vector of the present invention comprises a polyhedrin promoter or other baculovirus promoter operably linked to a nucleotide sequence comprising at least one cistron operably linked to a nucleotide sequence of an EV71, HCV, or EMCV IRES or a homolog, variant, or fragment thereof, or a variant or fragment of an EV71, HCV, or EMCV IRES homolog. The recombinant baculovirus transfer vector of the present invention may further comprise one or more additional “IRES-cistron” elements. Upon transfection of the recombinant transfer vector and baculovirus genomic DNA into susceptible host cells, the recombinant transfer vector and baculovirus genomic DNA undergo homologous recombination, thereby incorporating the gene(s) of interest into the baculovirus genome. Recombinant baculoviruses capable of expressing the gene(s) of interest are released into the extracellular medium. However, because neither transfection nor homologous recombination is 100% efficient, the result will be a mixture of cells that produce recombinant baculoviruses and those that do not. Recombinant baculoviruses capable of expressing the gene(s) of interest in baculovirus host cells are thereafter selected by appropriate screening or genetic selection techniques.

One means of selecting the recombinant baculovirus utilizes the plaque assay method. Plaque assays are designed to produce distinct viral plaques in a monolayer of host cells under conditions where each plaque is the result of a cell being infected by a single virus. Plaques are generated by infecting baculovirus host cells with diluted medium from cells transfected with the recombinant transfer vector and baculovirus genomic DNA. Infected cells form plaques, which may be visualized by overlaying infected cells with agar or under a microscope. Viral plaques may be isolated and are evaluated for recombinant baculovirus capable of expressing a gene of interest.

Many screening methods are available in the art to confirm that plaques isolated from the cotransfection contain recombinant baculoviruses. Preferred methods detect the synthesis of the target protein, e.g. Western blotting, ELISA, or biochemical assays for the expressed protein. Southern blot analysis and PCR may also confirm that the target gene is present in the recombinant baculovirus genome.

The present invention also relates to the treatment of a patient, or for the benefit of a patient, by administration of a nucleic acid vector or biological vector in an amount sufficient to direct the expression of a desired gene(s) in a patient. Administration of the nucleic acid vector or biological vector may provide the expression of a desired gene(s) that is deficient or non-functional in a patient. The nucleic acid vector or biological vector may be directly administered to a patient, for example, by intravenous or intramuscular injection or by aerosolization into the lungs. Alternatively, an ex vivo gene therapy protocol may be adopted, which comprises excising cells or tissues from a patient, introducing the nucleic acid vector or biological vector into the excised cells or tissues, and reimplanting the cells or tissues into the patient (see, for example, Knoell D. L., et al., (1998) Am. J. Health Syst. Pharm. 55:899-904; Raymon H. K., et al., (1997) Exp. Neurol. 144:82-91; Culver K. W., et al., (1990) Hum. Gene Ther. 1:399-410; Kasid A., et al., (1990) Proc. Natl. Acad. Sci. U.S.A. 87:473-477). The nucleic acid vector or biological vector may be introduced into excised cells or tissues by transfection or infection, such as by the methods described above.

A patient is hereby defined as any person or non-human animal in need of a specific protein, polypeptide, or peptide, or to any subject for whom treatment may be beneficial, including humans and non-human animals. Such non-human animals to be treated include all domesticated and feral vertebrates. One of skill in the art will, of course, recognize that the choice of protein, polypeptide, or peptide will depend on the disease or condition to be treated in a particular system.

The present invention further relates to a method of screening for anti-viral compounds capable of interfering with cap-independent translation from viral IRESs. Viral IRESs may function to support the infection, replication, and propagation of the virus in infected hosts through a cap-independent translation mechanism for essential viral proteins. Thus, the method of the present invention utilizes a multicistronic nucleic acid vector comprising a promoter operably linked to a nucleotide sequence comprising at least one cistron operably linked to a nucleotide sequence of a viral IRES or a homolog, variant, or fragment thereof, or a variant or fragment of a viral IRES homolog. The nucleic acid vector may further comprise one or more additional “IRES-cistron” elements in tandem for expression of at least two cistrons. The method comprises transfecting into a cell a multicistronic nucleic acid vector which directs the cap-independent translation of at least one recombinant protein from a viral IRES, or a homolog, variant, or fragment thereof, or a variant or fragment of a viral IRES homolog, contacting the transfected cell with a test compound, and detecting a decrease in recombinant protein production compared to a transfected cell without the test compound. A test compound may be any chemical, protein, peptide, polypeptide, or nucleic acid (DNA or RNA). The test compound may be naturally-occurring or may be synthesized by methods known in the art. In an embodiment of the present invention, the method of the present invention is used to screen for EV71, HCV, or EMCV anti-viral compounds.

The present invention is illustrated by the following Examples, which are not intended to be limiting in any way.

### EXAMPLE 1

The EMCV IRES has IRES Activity in Insect Cells

The EMCV IRES has been previously reported to be highly efficient in mammalian systems but inactive in insect cells (Finkelstein Y., et al., (1999) J. Biotech. 75:33-44). The inventors have surprisingly found that the EMCV IRES does function in insect cells.

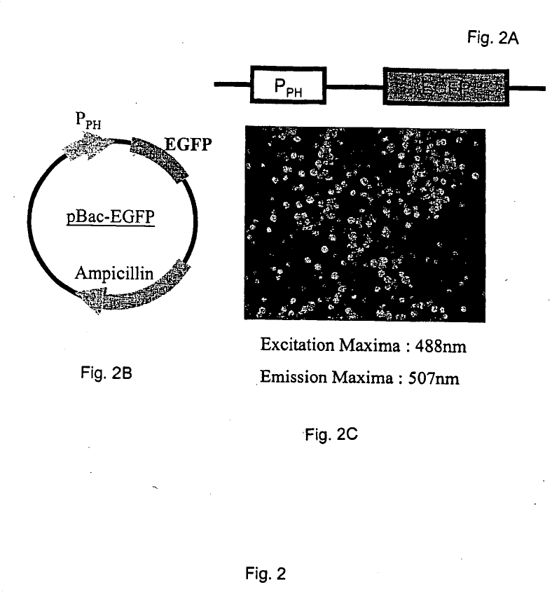

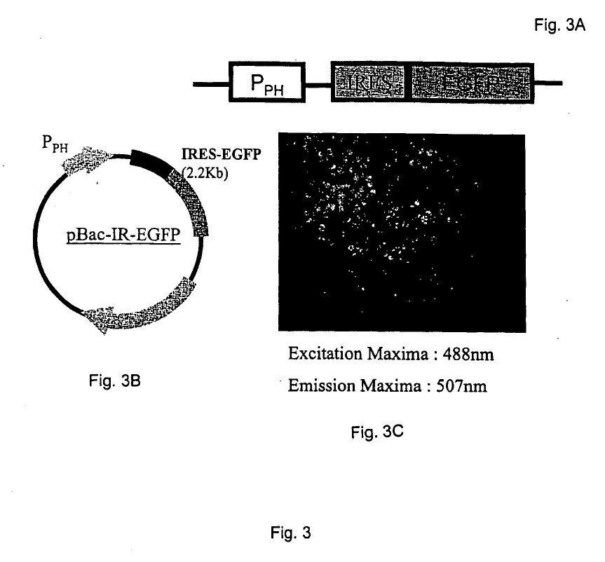

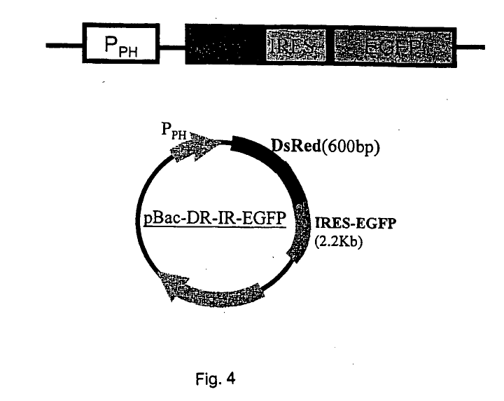

A recombinant baculovirus expression system was used to test for EMCV IRES activity in insect cells. Baculovirus transfer vectors were created using pBlueBac4.5 (Invitrogen). The enhanced green fluorescent protein (EGFP) coding sequence was inserted into the multiple cloning site of pBlueBac4.5 and placed under the control of the baculovirus polyhedrin promoter (P_{PH}). The resulting control vector was designated pBac-EGFP (FIGS. 2A and 2B). In another transfer vector, pBac-IR-EGFP, the EMCV IRES sequence (Jang, S. K., and E. Wimmer, (1990) Genes Dev. 4:1560-1572) was placed immediately in front of the EGFP coding sequence (FIGS. 3A and 3B). A bicistronic transfer vector carrying the cistrons for the red fluorescent protein from Discosoma sp. (DsRed) and EGFP were also created. In pBacDS-IRE-EGFP, the baculovirus polyhedrin promoter drives the mRNA synthesis of the nucleotide sequence containing the DsRed and EGFP genes. The EMCV IRES was inserted between the DsRed and EGFP genes (FIG. 4). It would be expected that the DsRed gene would be expressed by the cap-dependent mechanism and the EGFP would be expressed by the cap-independent mechanism driven by the EMCV IRES.

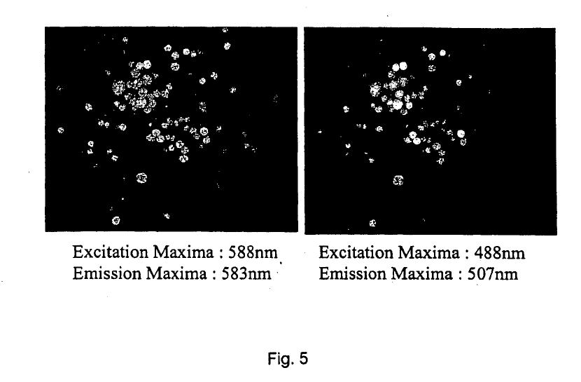

Recombinant baculoviruses were generated using the MaxBac 2.0 baculovirus expression system from Invitrogen. Baculovirus host insect cells, Sf9 cells, were infected with recombinant viruses carrying the pBac-EGFP, pBac-IR-EGFP, or pBacDs-IR-EGFP for 2 days, after which time the cells were analyzed by fluorescent microscopy for EGFP (excitation maxima 488 nm; emission maxima 507 nm) and/or DsRed (excitation maxima 588 nm; emission maxima 583 nm). As expected and shown in FIG. 2C, cells infected with the recombinant baculovirus carrying pBac-EGFP expressed EGFP by the cap-dependent mechanism. FIG. 3C shows that cells infected with the recombinant baculovirus carrying pBac-IR-EGFP was slightly less efficient in expressing EGFP, presumably because the presence of the EMCV IRES near the polyhedrin promoter interfered with cap-dependent translation of EGFP. Cells infected with the recombinant baculovirus carrying the bicistronic vector pBacDs-IR-EGFP expressed both DsRed (FIG. 5, left panel) and EGFP (FIG. 5, right panel) in the same cell. Thus, contrary to previous reports, EMCV IRES is capable of directing IRES-dependent translation of a recombinant protein in insect cells.

### EXAMPLE 2

The EV71, HCV, and EMCV IRESs are Active in a Wide Range of Cell Types

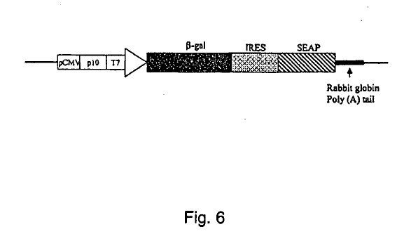

The EV71, HCV, and EMCV IRESs were analyzed for activity in various cell types, including insect cells (Sf9), mammalian cells (COS-7 and Huh7), and bacterial cells (BL21). The pTriEX-4 vector (Novagen) was used to generate bicistronic nucleic acid vectors for recombinant protein expression in all three cell types. The pTriEx-4 vector contains the cytomegalovirus (CMV) immediate early promoter, which is active in mammalian cells, the p10 promoter of the AcMNPV baculovirus, which is active in insect cells, and the T7 promoter from bacteriophage, which is active in bacterial cells. As depicted in FIG. 6, the β-galactosidase (β-gal) and secreted alkaline phosphatase (SEAP) genes were placed under the control of one of the three promoters present in pTriEX-4 for mRNA synthesis. The EV71 (FIG. 1), HCV (Tsukiyama-Kohara K., et al., (1992) J. Virol. 66:1476-1483), or EMCV IRES (Jang, S. K., and E. Wimmer, (1990) Genes Dev. 4:1560-1572) was inserted between the β-galactosidase and SEAP genes to drive the IRES-dependent expression of the SEAP gene, and the respective bicistronic nucleic acid vectors were designated pGS-EV71, pGS-HCV, and pGS-EMCV.

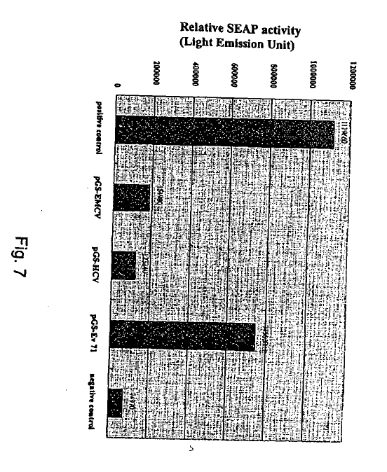

For detecting IRES activity in Sf9 insect cells, recombinant baculoviruses carrying pGS-EV71, pGS-HCV, or pGS-EV71 were generated according to the pTriEx System Manual (Novagen). Sf9 cells were infected with the recombinant baculoviruses and media of infected cells were harvested 72 hours after infection and analyzed for SEAP activity. As a positive control, a recombinant baculovirus was generated by recombining baculovirus genomic DNA with a recombinant transfer vector carrying the SEAP gene without any preceding IRES sequences in the pTriEX-4 vector. As a negative control, wild-type AcMNPV baculovirus was used to infect Sf9 cells. As shown in FIG. 7, EV71, HCV, and EMCV IRESs all had greater activity in Sf9 cells than the negative control. The EV71 IRES showed highest activity.

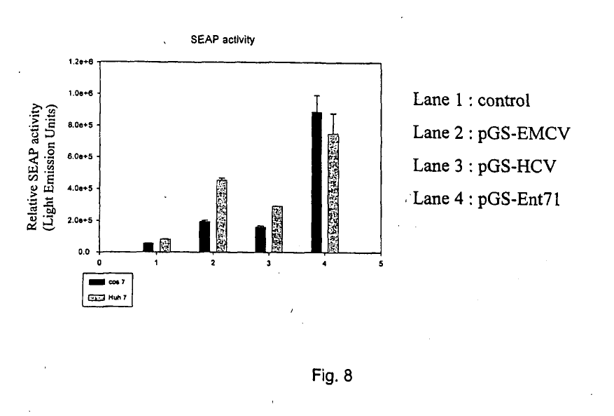

For testing IRES activity in mammalian cells, pGS-EMCV, pGS-HCV, and pGS-EV71 were transfected into COS-7 cells (a monkey kidney cell line) and Huh7 cells (a human hepatoma cell line) as outlined in the pTriEx System Manual (Novagen). In mammalian cells, mRNA from the nucleic acid vectors were generated from the CMV promoter. 48 hours after transfection, the media from transfected cells were assayed for SEAP activity. EV71, HCV, and EMCV IRESs all showed activity in both mammalian cell lines compared with the negative control, a monocistronic nucleic acid vector expressing the P-galactosidase gene under the control of the CMV promoter (pCMV-gal) (FIG. 8). The EV71 IRES again showed the highest activity in both mammalian cell lines.

For testing IRES activity in bacterial cells, pGS-EMCV, pGS-HCV, and pGS-EV71 were transformed into BL21 cells as outlined in the pTriEx System Manual (Novagen). In bacterial cells, mRNA from the nucleic acid vectors were generated from the T7 promoter, which may be induced with IPTG to generate high levels of mRNA.

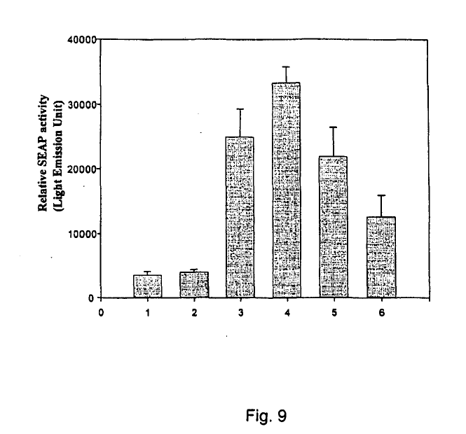

Cells were harvested three hours after induction with 0.4 mM IPTG and analyzed for SEAP activity. As shown in FIG. 9, EMCV IRES had high activity in bacterial cells without and with IPTG induction (lanes 3 and 4, respectively), compared with untransformed BL21 cells (land 1) and BL21 cells transformed with pTriEX-4 containing no reporter gene (lane 2). This is the first time that the EMCV IRES has been shown to have activity in bacterial cells. The HCV IRES and EV71 IRES also had activity in bacterial cells (lanes 5 and 6, respectively).

### EXAMPLE 3

Interferon-Alpha (IFN-α) Interferes with Cap-independent Translation from the EV71 and HCV IRES

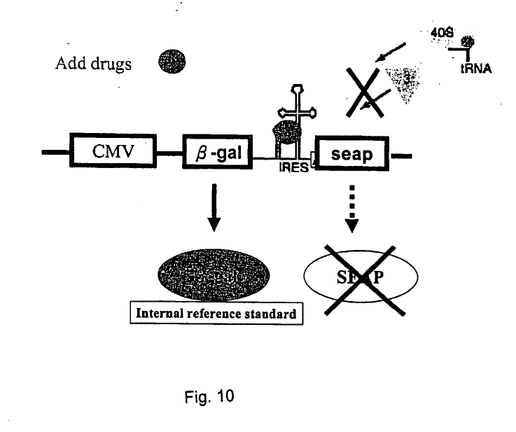

Bicistronic nucleic acid vectors containing the EV71 and HCV IRESs were utilized to screen for anti-viral compounds that are capable of interfering with cap-independent translation from the viral IRESs. Anti-viral compounds are expected to bind to the IRES and interfere with SEAP expression as depicted in FIG. 10. It has been shown by others that the first (cap-dependent) cistron paralleled the steady-state level of mRNA but was not significantly influenced by the protein coding sequence on the mRNA (Hennecke, M., et al., (2001) Nucleic Acids Res. 29:3327-3334). Therefore, translation from the cap-dependent cistron may be used as an internal standard to monitor for differences in mRNA levels.

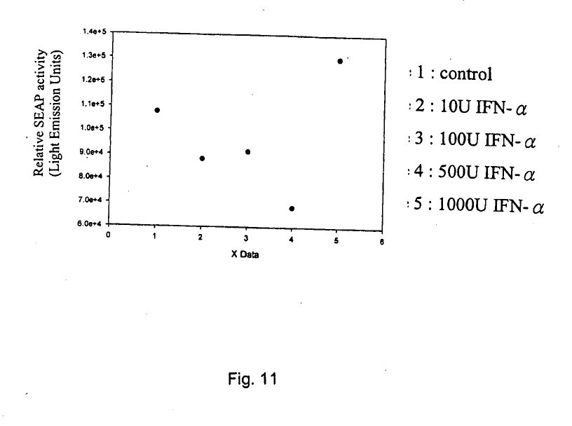

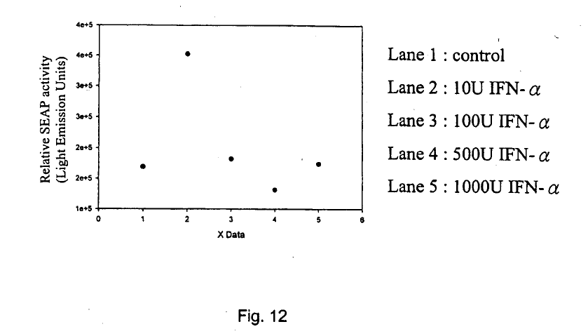

The bicistronic nucleic acid vectors, pGS-EV71 and pGS-HCV described in Example 2 were transfected into Huh7 cells and cultured in the presence of varying amounts of IFN-α. Media from transfected cells were harvested and analyzed for SEAP activity 48 hours after transfection. Control cells were transfected with the respective bicistronic nucleic acid vectors but cultured without IFN-α. As shown in FIGS. 11 and 12, 500 units of IFN-α inhibited both HCV and EV71 IRES activity, respectively.

The specification is most thoroughly understood in light of the teachings of the references cited within the specification, all of which are hereby incorporated by reference in their entirety. The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with the true scope and spirit of the invention being indicated by the following claims.

1. A nucleic acid vector for the expression of at least two cistrons comprising:
a. a promoter operably linked to a nucleotide sequence comprising at least two cistrons; and
b. at least one nucleotide sequence comprising an IRES selected from EV71, HCV, or EMCV, or a variant or fragment thereof, operably linked to at least one of said at least two cistrons, wherein said nucleotide sequence, or variant or fragment thereof, provides IRES activity.

2. The nucleic acid vector of claim 1, wherein at least one of said at least two cistrons comprises a reporter gene.

3. The nucleic acid vector of claim 1, wherein at least one of said at least two cistrons comprises a therapeutic gene.

4. A biological vector capable of expressing at least two cistrons comprising the nucleic acid vector of claim 1.

5. The biological vector of claim 4, wherein said biological vector is selected from poxvirus, adenovirus, herpesvirus, adeno-associated virus, retrovirus, and baculovirus.

6. A nucleic acid vector for the expression of at least two cistrons comprising:
a. a promoter operably linked to a nucleotide sequence comprising at least two cistrons; and
b. at least one nucleotide sequence comprising a homolog of an IRES selected from EV71, HCV, or EMCV, or a variant or fragment thereof, operably linked to at least one of said two cistrons, wherein said homolog, or a variant or fragment thereof, provides IRES activity.

7. The nucleic acid vector of claim 6, wherein at least one of said at least two cistrons comprises a reporter gene.

8. The nucleic acid vector of claim 6, wherein at least one of said at least two cistrons comprises a therapeutic gene.

9. A biological vector capable of expressing said at least two cistrons comprising the nucleic acid vector of claim 6.

10. The biological vector of claim 9, wherein said biological vector is selected from poxvirus, adenovirus, herpesvirus, adeno-associated virus, retrovirus, and baculovirus.

11. A host cell comprising the nucleic acid vector of claim 1.

12. The host cell of claim 11, wherein said host cell is an insect cell.

13. The host cell of claim 11, wherein said host cell is a mammalian cell.

14. The host cell of claim 11, wherein said host cell is a bacterial cell.

15. A host cell comprising the nucleic acid vector of claim 6.

16. The host cell of claim 15, wherein said host cell is an insect cell.

17. The host cell of claim 15, wherein said host cell is a mammalian cell.

18. The host cell of claim 15, wherein said host cell is a bacterial cell.

19. A method for expressing at least two cistrons comprising: introducing into a host cell: a nucleic acid vector comprising:
a. a promoter operably linked to a nucleotide sequence comprising at least two cistrons; and
b. at least one nucleotide sequence comprising an IRES selected from EV71, HCV, or EMCV, or a variant or fragment thereof operably linked to at least one of said at least two cistrons, wherein said nucleotide sequence, or variant or fragment thereof, provides IRES activity.

20. A method for expressing at least two cistrons comprising: introducing into a host cell: a nucleic acid vector comprising:
a. a promoter operably linked to a nucleotide sequence comprising at least two cistrons; and
b. at least one nucleotide sequence comprising a homolog of an IRES selected from EV71, HCV, or EMCV, or a variant or fragment thereof operably linked to at least one of said two cistrons, wherein said homolog, or variant or fragment thereof provides IRES activity.

21. A baculovirus transfer vector for the expression of at least two cistrons comprising:
a. a baculovirus promoter operably linked to a nucleotide sequence comprising at least two cistrons; and
b. at least one nucleotide sequence comprising an IRES selected from EV71, HCV, or EMCV, or a variant or fragment thereof, operably linked to at least one of said at least two cistrons, wherein said nucleotide sequence, or variant or fragment thereof provides IRES activity.

22. The baculovirus transfer vector of claim 21, wherein at least one of at least two cistrons comprises a reporter gene.

23. The baculovirus transfer vector of claim 21, wherein at least one of at least two cistrons comprises a therapeutic gene.

24. A recombinant baculovirus capable of expressing at least two cistrons in a host cell comprising a baculovirus genome comprising:
a. a baculovirus promoter operably linked to a nucleotide sequence comprising at least two cistrons; and
b. at least one nucleotide sequence comprising an IRES selected from EV71, HCV, or EMCV, or a variant or fragment thereof operably linked to at least one of said at least two cistrons, wherein said nucleotide sequence, or variant or fragment thereof, provides IRES activity.

25. A method for producing a recombinant baculovirus capable of expressing at least two cistrons comprising:
a. introducing a baculovirus transfer vector of claim 21 and a baculovirus genomic DNA into a baculovirus host cell so as to effect homologous recombination; and
b. isolating a recombinant baculovirus.

26. A baculovirus host cell expressing at least two cistrons comprising the recombinant baculovirus of claim 24.

27. A baculovirus transfer vector for the expression of at least two cistrons comprising:
a. a baculovirus promoter operably linked to a nucleotide sequence comprising at least two cistrons; and
b. at least one nucleotide sequence comprising a homolog of an IRES selected from EV71, HCV, or EMCV, or a variant or fragment thereof, operably linked to at least one of said at least two cistrons, wherein said nucleotide sequence, or variant or fragment thereof provides IRES activity.

28. The baculovirus transfer vector of claim 27, wherein at least one of at least two cistrons comprises a reporter gene.

29. The baculovirus transfer vector of claim 27, wherein at least one of at least two cistrons comprises a therapeutic gene.

30. A recombinant baculovirus capable of expressing at least two cistrons in a host cell comprising a baculovirus genome comprising:
a. a baculovirus promoter operably linked to a nucleotide sequence comprising at least two cistrons; and
b. at least one nucleotide sequence comprising a homolog or an IRES selected from EV71, HCV, or EMCV, or a variant or fragment thereof operably linked to at least one of said at least two cistrons, wherein said nucleotide sequence, or variant or fragment thereof, provides IRES activity.

31. A method for producing a recombinant baculovirus capable of expressing at least two cistrons comprising:
a. introducing a baculovirus transfer vector of claim 27 and a baculovirus genomic DNA into a baculovirus host cell so as to effect homologous recombination; and
b. isolating a recombinant baculovirus.

32. A baculovirus host cell expressing at least two cistrons comprising the recombinant baculovirus of claim 30.

33. A kit for recombinant protein expression in bacteria, insect, and/or mammalian cells comprising at least one nucleic acid vector comprising at least one IRES sequence functional in a bacterial cell, at least one nucleic acid vector comprising at least one IRES sequence functional in a insect cell, and at least one nucleic acid vector comprising at least one IRES sequence functional in a mammalian cell.

34. The kit of claim 33, wherein said at least one nucleic acid vector comprises at least one IRES sequence selected from EV71, HCV, or EMCV.

35. The kit of claim 33, wherein the kit comprises a single nucleic acid vector comprising at least one IRES sequence functional in a bacteria, insect, and mammalian cell.

36. The kit of claim 33, wherein the kit comprises two nucleic acid vectors wherein said two nucleic acid vectors each comprise at least one IRES sequence functional in bacteria, insect, and/or mammalian cells.

37. A method of treating a patient comprising administering the nucleic acid vector of claim 1 or 6.

38. A method of treating a patient comprising administering the biological vector of claim 4 or 9.

39. A method of treating a patient comprising:
a. excising a cell or tissue from said patient;
b. introducing the nucleic acid vector of claim 1 or 6 into said excised cell or tissue; and
c. reimplanting said cell or tissue into said patient.

40. A method of treating a patient comprising:
a. excising a cell or tissue from said patient;
b. introducing the biological vector of claim 4 or 9 into said excised cell or tissue; and
c. reimplanting said cell or tissue into said patient.

41. A method for screening for an anti-viral compound capable of interfering with cap-independent translation from an IRES selected from EV71, HCV, or EMCV comprising:
a. transfecting into a cell the nucleic acid vector of claim 1 or 6;
b. contacting said transfected cell with a test compound; and
c. detecting a decrease in recombinant protein production compared to a transfected cell without the test compound.


SEQUENCE LISTING (1 sequence)

SEQ ID NO: 1 - DNA, 709 bp, Enterovirus 71
ccactgggccgctagcactctggtactgaggtacctttgtgcgcctgtttttactccccttccccccgaagtaacttagaagctgtaaatcagatcaatagcaggtgtggcacaccagtcatacctcgatcaagcacttctgtttccccggactgagtatcaataggctgctcgcgcggctgaaggagaaaacgttcgttacccgaccaactacttcgagaagcttagtaccaccatgaacgaggcagggtgtttcgctcagcacaaccccagtgtagatcaggctgatgagtcactgcaacccccatgggcgaccatggcagtggctgcgttggcggcctgcccatggagaaatccatgggacgctctaattctgacatggtgtgaagagcctattgagctagctggtagtcctccggcccctgaatgcggctaatcccaactgcggagcacatgctcacaaaccagtgggtggtgtgtcgtaacgggcaactctgcagcggaaccgactactttgggtgtccgtgtttccttttattcttatattggctgcttatggtgacaatcaaagagttgttaccatatagctattggattggccatccggtgtgcaacagggcaattgtttacctatttattggttttgtaccattatcactgaagtctgtgatcactctcaaattcattttgaccctcaacataatcagac